(19) United States
(12) Patent Application Publication
(10) Pub. No.: US 2025/0134790 A1
(43) Pub. Date: May 1, 2025

(54) ANTIMICROBIAL COMPOSITIONS

(71) Applicant: Firmenich SA, Satigny (CH)

(72) Inventors: Yang HUANG, Shanghai (CN); Qiu-Min MA, Shanghai (CN); Wolfgang FIEBER, Satigny (CH)

(21) Appl. No.: 18/681,256

(22) PCT Filed: Aug. 11, 2022
(86) PCT No.: PCT/EP2022/072527

§ 371 Date: Feb. 5, 2024

(30) Foreign Application Priority Data
Aug. 17, 2021 (WO) PCT/CN21/112970
Sep. 29, 2021 (EP) 21199840.6

(51) Int. Cl. A61K 8/49; A01N 25/30; A01N 31/02; A01N 35/04; A01N 37/02; A01N 37/34; A01N 43/08; A01N 65/10; A01N 65/22; A01N 65/24; A01N 65/36; A01P 1/00; A61K 8/33; A61K 8/34; A61K 8/37; A61K 8/40; A61K 8/44; A61K 8/46; A61K 8/60; A61K 8/9789; A61Q 17/00; A61Q 19/10

(52) U.S. Cl. CPC A61K 8/4973; A01N 25/30; A01N 31/02; A01N 35/04; A01N 37/02; A01N 37/34; A01N 43/08; A01N 65/10; A01N 65/22; A01N 65/24; A01N 65/36; A01P 1/00; A61K 8/33; A61K 8/342; A61K 8/37; A61K 8/40; A61K 8/44; A61K 8/463; A61K 8/602; A61K 8/9789; A61Q 17/005; A61Q 19/10

(57) ABSTRACT

Described herein are methods and compositions that can be applied to personal cleaning, oral care, deodorant and hard surface cleaning applications, including liquid soap, foaming soap, liquid dishwashing detergent, shower gel, shampoo, emulsified deodorant, mouthwash, toothpaste and facial cleanser, in order to reduce or eliminate microbes on a surface or body part.

### FIELD OF THE INVENTION

The present invention is related to methods and compositions for human or animal body surface and hair cleaning applications, soft and porous substrates like fabric or hard surfaces cleaning applications, deodorant, oral care, or air care applications in order to reduce or eliminate microbes.

### BACKGROUND

Perfume ingredients may have antimicrobial activity. However, the antimicrobial activity of a perfume ingredient can be largely impacted by the application bases, specifically, the surfactant bases, e.g., liquid soaps or shampoo. In these bases, perfume ingredients are incorporated into surfactant micelles and the bioavailability of the perfume ingredients is significantly reduced. Therefore, much higher concentrations of perfume ingredients are needed, but are not acceptable in terms of sensory affect, cost, and solubility issues. These obstacles are overcome by the compositions, methods, and uses of the present invention.

### SUMMARY OF THE INVENTION

The present invention encompasses methods and antimicrobial compositions comprising a perfume ingredient and a surfactant and a hydrotrope, wherein the perfume ingredient has a bactericidal effect of a 5.5 log reduction in an ethanol solution at a concentration of less than or equal to 0.1% in ethanol solution.

In an embodiment of the invention the perfume ingredient is 1-phenylethyl acetate, 1-octanol, (1,3,3-trimethylbicyclo[2.2.1]heptan-2-ol, undecanal, (2E)-2-methyl-3-phenyl-2-propenal, (Z)-2-nonenal, 1-(5-propyl-1,3-benzodioxol-2-yl)ethenone, (2E,6Z)-2,6-nonadien-1-ol, ethyl benzoate, 1,7,7-trimethylbicyclo[2.2.1]heptan-2-ol, benzyl butanoate, 1-butoxycarbonylethyl butanoate, ethyl hexanoate, 5-isopropyl-2-methylphenol, (4E,8E)-4,8-cyclododecadien-1-one, (4E,8Z)-4,8-cyclododecadien-1-one, (4Z,8E)-4,8-cyclododecadien-1-one, methyl (2E)-2-methyl-2-hexenoate, ethyl (E)-3-phenyl-2-propenoate, methyl (E)-3-phenyl-2-propenoate, (Z)-3,7-dimethyl-2,6-octadienal, (E)-3,7-dimethyl-2,6-octadienal, 3,5,5-trimethyl-1-hexanol, 3-[4-methyl-3-cyclohexen-1-yl]-1-butanol, (2E)-2-ethyl-4-[2,2,3-trimethyl-3-cyclopenten-1-yl]-2-buten-1-ol, 4-decanolide, (Z)-4-decenal, 2-methoxy-4-propylphenol, 2,6-dimethyl-7-octen-4-one, 2,6-dimethyl-4-heptanol, (E)-3,7-dimethyl-2,6-octadien-1-ol, 2,4,6-trimethyl-3-cyclohexene-1-methanol, 2-methoxy-4-[(1E)-1-propen-1-yl]phenol, (2E)-2-methyl-3-(4-methylphenyl)-2-propen-1-ol, 2,5-dimethyl-2-indanmethanol, 1,2-dimethoxy-4-[(1Z)-1-propen-1-yl]benzene, 1,2-dimethoxy-4-[(1E)-1-propen-1-yl]benzene, (4E)-4-methyl-5-(4-methylphenyl)-4-pentenal, (2,2-dimethyl-3-[(2Z)-3-methyl-2,4-pentadien-1-yl]oxirane, (2,2-dimethyl-3-[(2E)-3-methyl-2,4-pentadien-1-yl]oxirane, 2-ethyl-1-hexanol, (Z)-3,7-dimethyl-2,6-octadien-1-ol, 1-(3-methyl-1-benzofuran-2-yl)ethenone, (Z)-6-nonen-1-ol, (2E,6Z)-2,6-nonadienal, 1,8-p-menthadien-7-ol, 3-methyl-5-phenyl-1-pentanol, 2,6,6-trimethyl-1,3-cyclohexadiene-1-carbaldehyde, ethyl 2-hydroxybenzoate, 2-(5,5,6-trimethylbicyclo[2.2.1]hept-2-yl)-1-cyclohexanol, 3-(5,5,6-trimethylbicyclo[2.2.1]hept-2-yl)-1-cyclohexanol, 4-(5,5,6-trimethylbicyclo[2.2.1]hept-2-yl)-1-cyclohexanol, 2-(1,7,7-trimethylbicyclo[2.2.1]hept-2-yl)-1-cyclohexanol, 3-methylindole, 8-mercapto-3-p-menthanone, 3,7-dimethyl-3-octanol, 1,3,3-trimethylbicyclo[2.2.1]heptan-2-one, 1-isopropyl-4-methylbicyclo[3.1.0]hexan-3-one, 1-isopropyl-4-methylbicyclo[3.1.0]hexan-3-one, thymol, 6-hexyltetrahydro-2H-pyran-2-one, 3-propylphenol, 2-cyclohexylethyl acetate, octanal, 1,3-nonanediyl diacetate, tetrahydro-3-pentyl-4(2H)-pyranyl acetate, 5-heptyldihydro-2(3H)-furanone, 3-butylidene-1-benzo[C] furanone, 3,7-dimethyl-6-octen-1-ol, 3,7-dimethyl-6-octenenitrile, coriander oil, cyclopropylmethyl (3Z)-3-hexenoate, cyclopropylmethyl (3E)-3-hexenoate, 3,7-dimethyl-1-octen-3-ol, cinnamon leaf oil, 4-decanolide, 3,5,6-trimethyl-3-cyclohexene-1-carbaldehyde, 2,4,6-trimethyl-3-cyclohexene-1-carbaldehyde, Lavandin Sumian essential oil, peppermint oil, 3,6,7-trimethyl-2,6-octadienal, petitgrain paraguay essential oil, (2Z)-4,8-dimethyl-2,7-nonadien-4-ol or a combination thereof

In a further embodiment of the invention the perfume ingredient is (4E,8E)-4,8-cyclododecadien-1-one, (4E,8Z)-4,8-cyclododecadien-1-one, (4Z,8E)-4,8-cyclododecadien-1-one, (4E)-4-methyl-5-(4-methylphenyl)-4-pentenal, 3,7-dimethyl-6-octenenitrile, 3,7-dimethyl-6-octen-1-ol, (2Z)-4,8-dimethyl-2,7-nonadien-4-ol, 1,3-nonanediyl diacetate, tetrahydro-3-pentyl-4(2H)-pyranyl acetate, (Z)-4-decenal, 3-[4-methyl-3-cyclohexen-1-yl]-1-butanol, undecanal, (2E)-2-ethyl-4-[2,2,3-trimethyl-3-cyclopenten-1-yl]-2-buten-1-ol, 3,7-dimethyl-3-octanol, 2-(5,5,6-trimethylbicyclo[2.2.1]hept-2-yl)-1-cyclohexanol, 3-(5,5,6-trimethylbicyclo[2.2.1]hept-2-yl)-1-cyclohexanol, 4-(5,5,6-trimethylbicyclo[2.2.1]hept-2-yl)-1-cyclohexanol, 2-(1,7,7-trimethylbicyclo[2.2.1]hept-2-yl)-1-cyclohexanol, 2-cyclohexylethyl acetate, 1,3,3-trimethylbicyclo[2.2.1]heptan-2-one, 1-isopropyl-4-methylbicyclo[3.1.0]hexan-3-one or a combination thereof.

In an embodiment of the invention the perfume ingredient is cyclopropylmethyl (3Z)-3-hexenoate or cyclopropylmethyl (3E)-3-hexenoate, 3,6,7-trimethyl-2,6-octadienal, (2Z)-4,8-dimethyl-2,7-nonadien-4-ol, octanal, 5-heptyldihydro-2(3H)-furanone, 3,7-dimethyl-6-octen-1-ol, 3,7-dimethyl-6-octenenitrile, 3,7-dimethyl-1-octen-3-ol, cinnamon leaf oil, Lavandin Sumian essential oil, 2-cyclohexylethyl acetate, peppermint oil, coriander oil, Petitgrain paraguay essential oil, 3-butylidene-1-benzo[C] furanone or a combination thereof.

A perfume ingredient in a composition of the invention may be present in an amount effective to provide an antimicrobial effect.

In aspects of the present invention, the perfume ingredient is at least 0.001% (w/v) of the antimicrobial composition.

A surfactant may be 0.1% to 30% (w/w) of the antimicrobial composition. In certain aspects, the surfactant is 0.1% to 20% (w/w) of the antimicrobial composition. In further aspects, the surfactant is 0.1% to 10% (w/w) of the antimicrobial composition. In a further aspect the surfactant is 9.1% to 30% (w/w) of the antimicrobial composition. In a further aspect the surfactant is 9.1% to 20% (w/w) of the antimicrobial composition. In a further aspect the surfactant is 9.1% to 10% (w/w) of the antimicrobial composition.

A surfactant of the present invention is may be an anionic surfactant, a non-ionic surfactant, an amphoteric surfactant or a combination thereof.

The anionic surfactant may be, for example, sodium lauryl ether sulfate, and the amphoteric surfactant may be cocamidopropyl betaine. The non-ionic surfactant may be an alkyl polyglycoside. A non-ionic surfactant according to the present invention may be coco-glucoside.

In aspects of the present invention, the antimicrobial composition may further include a hydrotrope. In further aspects, the antimicrobial composition comprises an additional agent active against gram-positive or gram-negative bacteria. In other aspects, the antimicrobial composition may further comprise a chelating agent selected from the group consisting of EDTA, and CDTA, and a combination thereof.

An antimicrobial composition of the present invention further comprises a hydrotrope.

In aspects of the present invention, the hydrotrope is toluene sulfonate, xylene sulfonate, cumene sulfonate, diisobutyl sulfosuccinate, or a combination thereof. In further aspects, the hydrotrope is a sodium, ammonium or potassium salt of a hydrotrope selected from toluene sulfonate, xylene sulfonate, cumene sulfonate, diisobutyl sulfosuccinate, sodium salicylate, sodium acetate and sodium benzoate dipropyleneglycol n-butyl ether; or a combination thereof.

In aspects of the present invention, an antimicrobial fragrance contains at least 25% (w/v) of the perfume ingredient. That is, the perfume ingredients having a bactericidal effect represent at least 25% (w/v) of the total fragrance mixture added to the composition.

In aspects of the present invention, the composition is preferably used for reducing or eliminating microbes on external surface of human or animal body or soft and porous substrates like fabric or for hard surfaces, or for deodorant, air care, oral care and hair care applications.

The present invention encompasses a consumer product comprising antimicrobial compositions according to the present invention, wherein the consumer product is a hair care product, a body care product, a skin care product, an oral care product, a female care product, a home care product, a laundry care product, or a body cleansing product, including but not limited to shampoo, shower gel, facial cleanser, shaving gel, liquid hand soap, foaming soap, hand sanitizer, soap bar, mouthwash, toothpaste, female hygiene composition, fabric cleanser, carpet cleanser, all purpose cleanser, dishwashing detergent, fresh produce detergent, deodorant, air fresher and air disinfectant.

The present invention encompasses methods for eliminating or/and reducing the number of microbes on a surface or body part, comprising contacting the surface or body part with the antimicrobial composition of the present invention.

Further, the present invention includes use of an antimicrobial composition of the present invention for eliminating or reducing the number of microbes on a surface or body part.

In aspects of the present invention, the antimicrobial composition may be combined with a malodor neutralizing system.

The malodor neutralizing system may be (a) a composition comprising at least one ingredient selected from: (i) at least one aldehyde of formula R1CHO, wherein R1 is an aliphatic linear or branched, saturated or unsaturated carbon chain containing from 1 to 12 carbon atoms; (ii) at least one ketone of formula R2COR3, wherein R2 is an ethyl or methyl group and R3 is an aliphatic linear or branched, saturated or unsaturated carbon chain containing from 1 to 12 carbon atoms; and (iii) primary alcohols of formula R4CH2OH, wherein R4 is an aliphatic linear or branched, saturated or unsaturated carbon chain containing 1 to 12 carbon atoms, optionally substituted with an aromatic moiety; and (b) a composition comprising: (i) at least one ingredient selected from the group consisting of (2E)-1-(2,6,6-trimethyl-2-cyclohexen-1-yl)-2-buten-1-one, (2E)-1-(2,6,6-trimethyl-1-cyclohexen-1-yl)-2-buten-1-one, (2E)-1-(2,2-dimethyl-6-methylenecyclohexyl)-2-buten-1-one, (E)-1-(2,6,6-trimethyl-3-cyclohexen-1-yl)-2-buten-1-one, 1-(5,5-dimethyl-1-cyclohexen-1-yl)-4-penten-1-one, (+−)-methyl-2,2-dimethyl-6-methylene-1-cyclohexanecarboxylate, α- or β-(E)-4-(2,6,6-trimethyl-2-cyclohexen-1-yl)-3-buten-2-one (α- or β-ionone), (1E)-1-(2,6,6-trimethyl-2-cyclohexen-1-yl)-1-penten-3-one, (1E)-1-(2,6,6-trimethyl-1-cyclohexen-1-yl)-1-penten-3-one, (E)-3-methyl-4-(2,6,6-trimethyl-2-cyclohexen-1-yl)-3-buten-2-oneγ-methyl-ionone, 1-(2,6,6-trimethyl-1 (2)-cyclohexen-1-yl)-1,6-heptadien-3-one and 1-(4,6,6-trimethyl-1,3-cyclohexadien-1-yl)-2-buten-1-one; and (ii) at least one nitrile ingredient selected from the group consisting of 3-phenyl-2-propenenitrile, 3 (E/Z)-3-methyl-5-phenyl-2-pentenenitrilecitronitrile, 3,7-dimethyl-6-octenenitrilecitronellyl nitrile, 2-propyl-1-heptanenitrile, dodecanenitrile, and the mixture of 3-(2,3-dimethyl-2 (3)-cyclopenten-1-yl)butanenitrile and 3-(2-methyl-3-methylene-1-cyclopentyl)butanenitrile; and c) a combination of a) and b).

An antimicrobial composition of the present invention may be combined with a malodor antagonist system. In aspects of the present invention, the antimicrobial composition may be combined with at least one compound that inhibits the activity of at least one olfactory receptor selected from the group consisting of: a DMTS olfactory receptor, an indole/skatole olfactory receptor, a butyric acid olfactory receptor, and a p-cresol olfactory receptor. The at least one compound that inhibits the activity of at least one olfactory receptor may be benzyl acetate, (1R,2R)-1,7,7-trimethyl-bicyclo[2.2.1]hept-2-yl acetate (isobornyl acetate), undec-10-enal, undec-9-enal, Virginia cedarwood essential oil, 3,7-dimethyl-2,6-octadienal, 3,7-Dimethyl-6-octen-1-ol, 3,7-dimethyloct-6-enenitrile, coumarin, (E)-1-(2,6,6-trimethyl-3-cyclohexen-1-yl)-2-buten-1-one, (E)-3-methyl-4-(2,6,6-trimethyl-2-cyclohexen-1-yl)-3-buten-2-onemethyl ionone gamma, (Z)-3,4,5,6,6-pentamethylhept-3-en-2-one, 2,6-dimethylhept-5-enal, menthone, 1-(5,5-dimethyl-1-cyclohexenyl)pent-4-en-1-one, patchouli essential oil, 2,6-nonadienal, (2-tert-butylcyclohexyl) acetate, 2-methyl-3-hexanone oxime (vertoxime), or 2-Methoxynaphthalene.

### DETAILED DESCRIPTION

According to the present invention, methods and antimicrobial compositions are provided that include a perfume ingredient and a surfactant and a hydrotrope, the perfume ingredient having a bactericidal effect of 5.5 log reduction in ethanol solution at a concentration of less than or equal to 0.1% in ethanol concentration,

A “perfume ingredient” as used herein, means a compound able to impart or modify, in a pleasant or positive way, the odor of a composition. In general terms, a perfume ingredient may belong to chemical classes as varied as alcohols, lactones, aldehydes, ketones, esters, ethers, acetates, nitriles, terpenoids, nitrogenous or sulphurous heterocyclic compounds and essential oils.

Another object of the invention is a perfuming composition comprising at least one ingredient selected from the group consisting of perfuming co-ingredients, a perfumery carrier and mixtures thereof, and optionally at least one perfumery adjuvant.

As liquid perfumery carrier one may cite, as non-limiting examples, a solubilizer or a solvent commonly used in perfumery. A detailed description of the nature and type of solvents commonly used in perfumery cannot be exhaustive. However, one can cite as non-limiting examples solvents such as dipropyleneglycol, diethyl phthalate, isopropyl myristate, benzyl benzoate, 2-(2-ethoxyethoxy)-1-ethanol or ethyl citrate, which are the most commonly used. For the compositions which comprise both a perfumery carrier and a perfumery co-ingredient, other suitable perfumery carriers than those previously specified, can be also ethanol, limonene or other terpenes, isoparaffins such as those known under the trademark Isopar® (origin: Exxon Chemical) or glycol ethers and glycol ether esters such as those known under the trademark Dowanol® (origin: Dow Chemical Company). By “perfumery co-ingredient” it is meant here a compound, which is used in a perfuming preparation or a composition to impart a hedonic effect and which is not a microcapsule as defined above. In other words such a co-ingredient, to be considered as being a perfuming one, must be recognized by a person skilled in the art as being able to impart or modify in a positive or pleasant way the odor of a composition, and not just as having an odor.

Non-limiting examples include:
Aldehydic ingredients: decanal, dodecanal, 2-methyl-undecanal, 10-undecenal, octanal and/or nonenal;
Aromatic-herbal ingredients: eucalyptus oil, camphor, eucalyptol, menthol and/or alpha-pinene;
Balsamic ingredients: coumarine, ethylvanillin and/or vanillin;
Citrus ingredients: dihydromyrcenol, 3,7-dimethylocta-2,6-dienal, orange oil, linalyl acetate, (−)-(R)-3,7-dimethyl-6-octenenitrile, orange terpenes, limonene, 1-P-menthen-8-yl acetate and/or 1,4(8)-P-menthadiene;
Floral ingredients: Methyl dihydrojasmonate, linalool, Citronellol, phenylethanol, 3-(4-tert-butylphenyl)-2-methylpropanal, hexylcinnamic aldehyde, benzyl acetate, benzyl salicylate, tetrahydro-2-isobutyl-4-methyl-4 (2H)-pyranol, beta ionone, methyl 2-(methylamino) benzoate, (E)-3-methyl-4-(2,6,6-trimethyl-2-cyclohexen-1-yl)-3-buten-2-one, hexyl salicylate, 3,7-dimethyl-1,6-nonadien-3-ol, 3-(4-isopropylphenyl)-2-methylpropanal, verdyl acetate, geraniol, P-menth-1-en-8-ol, 4-(1,1-dimethylethyl)-1-cyclohexyle acetate, 1,1-dimethyl-2-phenylethyl acetate, 4-cyclohexyl-2-methyl-2-butanol, amyl salicylate, high cis methyl dihydrojasmonate, 3-methyl-5-phenyl-1-pentanol, verdyl proprionate, geranyl acetate, tetrahydro linalool, cis-7-P-menthanol, Propyl (S)-2-(1,1-dimethylpropoxy)propanoate, 2-methoxynaphthalene, 2,2,2-trichloro-1-phenylethyl acetate, 4/3-(4-hydroxy-4-methylpentyl)-3-cyclohexene-1-carbaldehyde, amylcinnamic aldehyde, 4-phenyl-2-butanone, isononyle acetate, 4-(1,1-diméthyléthyl)-1-cyclohexyl acetate, verdyl isobutyrate and/or mixture of methylionones isomers;
Fruity ingredients: gamma undecalactone, 4-decanolide, ethyl 2-methyl-pentanoate, hexyl acetate, ethyl 2-methylbutanoate, gamma nonalactone, allyl heptanoate, 2-phenoxyethyl isobutyrate, ethyl 2-methyl-1,3-dioxolane-2-acetate and/or diethyl 1,4-cyclohexane dicarboxylate;
Green ingredients: 2,4-Dimethyl-3-cyclohexene-1-carbaldehyde, 2-tert-butyl-1-cyclohexyl acetate, (+−)-1-phenylethyl acetate, allyl (2-methylbutoxy) acetate, 4-methyl-3-decen-5-ol, diphenyl ether, (Z)-3-hexen-1-ol and/or 1-(5,5-dimethyl-1-cyclohexen-1-yl)-4-penten-1-one;
Musk ingredients: 1,4-dioxa-5,17-cycloheptadecanedione, pentadecenolide, 3-Methyl-5-cyclopentadecen-1-one, 1,3,4,6,7,8-hexahydro-4,6,6,7,8,8-hexamethyl-cyclopenta-g-2-benzopyrane, (1S,1′R)-2-[1-(3′,3′-dimethyl-1′-cyclohexyl)ethoxy]-2-methylpropyl propanoate, pentadecanolide and/or (1S,1′R)-[1-(3′,3′-Dimethyl-1′-cyclohexyl)ethoxycarbonyl]methyl propanoate;
Woody ingredients: 1-(octahydro-2,3,8,8-tetramethyl-2-naphtalenyl)-1-ethanone, patchouli oil, terpenes fractions of patchouli oil, (1′R,E)-2-ethyl-4-(2′,2′,3′-trimethyl-3′-cyclopenten-1′-yl)-2-buten-1-ol, 2-ethyl-4-(2,2,3-trimethyl-3-cyclopenten-1-yl)-2-buten-1-ol, Methyl cedryl ketone, 5-(2,2,3-trimethyl-3-cyclopentenyl)-3-methylpentan-2-ol, 1-(2,3,8,8-tetramethyl-1,2,3,4,6,7,8,8a-octahydronaphthalen-2-yl)ethan-1-one and/or isobornyl acetate;
Other ingredients (e.g. amber, powdery spicy or watery): dodecahydro-3a,6,6,9a-tetramethyl-naphtho[2,1-b]furan and any of its stereoisomers, heliotropin, anisic aldehyde, eugenol, cinnamic aldehyde, clove oil, 3-(1,3-benzodioxol-5-yl)-2-methylpropanal and/or 3-(3-isopropyl-1-phenyl)butanal.

Perfume ingredients are not limited to those above. Perfume ingredients may also be found in reference texts such as the book by S. Arctander, Perfume and Flavor Chemicals, 1969, Montclair, New Jersey, USA, or its more recent versions, or in other works of a similar nature, as well as in the patent literature in the field of perfumery.

In a further embodiment of the invention the perfume ingredient is 1-phenylethyl acetate, 1-octanol, (1,3,3-trimethylbicyclo[2.2.1]heptan-2-ol, undecanal, (2E)-2-methyl-3-phenyl-2-propenal, (Z)-2-nonenal, 1-(5-propyl-1,3-benzodioxol-2-yl)ethenone, (2E,6Z)-2,6-nonadien-1-ol, ethyl benzoate, 1,7,7-trimethylbicyclo[2.2.1]heptan-2-ol, benzyl butanoate, 1-butoxycarbonylethyl butanoate, ethyl hexanoate, 5-isopropyl-2-methylphenol, (4E,8E)-4,8-cyclododecadien-1-one, (4E,8Z)-4,8-cyclododecadien-1-one, (4Z,8E)-4,8-cyclododecadien-1-one, methyl (2E)-2-methyl-2-hexenoate, ethyl (E)-3-phenyl-2-propenoate, methyl (E)-3-phenyl-2-propenoate, (Z)-3,7-dimethyl-2,6-octadienal, (E)-3,7-dimethyl-2,6-octadienal, 3,5,5-trimethyl-1-hexanol, 3-[4-methyl-3-cyclohexen-1-yl]-1-butanol, (2E)-2-ethyl-4-[2,2,3-trimethyl-3-cyclopenten-1-yl]-2-buten-1-ol, 4-decanolide, (Z)-4-decenal, 2-methoxy-4-propylphenol, 2,6-dimethyl-7-octen-4-one, 2,6-dimethyl-4-heptanol, (E)-3,7-dimethyl-2,6-octadien-1-ol, 2,4,6-trimethyl-3-cyclohexene-1-methanol, 2-methoxy-4-[(1E)-1-propen-1-yl]phenol, (2E)-2-methyl-3-(4-methylphenyl)-2-propen-1-ol, 2,5-dimethyl-2-indanmethanol, 1,2-dimethoxy-4-[(1Z)-1-propen-1-yl]benzene, 1,2-dimethoxy-4-[(1E)-1-propen-1-yl]benzene, (4E)-4-methyl-5-(4-methylphenyl)-4-pentenal, (2,2-dimethyl-3-[(2Z)-3-methyl-2,4-pentadien-1-yl]oxirane, (2,2-dimethyl-3-[(2E)-3-methyl-2,4-pentadien-1-yl]oxirane, 2-ethyl-1-hexanol, (Z)-3,7-dimethyl-2,6-octadien-1-ol, 1-(3-methyl-1-benzofuran-2-yl)ethenone, (Z)-6-nonen-1-ol, (2E,6Z)-2,6-nonadienal, 1,8-p-menthadien-7-ol, 3-methyl-5-phenyl-1-pentanol, 2,6,6-trimethyl-1,3-cyclohexadiene-1-carbaldehyde, ethyl 2-hydroxybenzoate, 2-(5,5,6-trimethylbicyclo[2.2.1]hept-2-yl)-1-cyclohexanol, 3-(5,5,6-trimethylbicyclo[2.2.1]hept-2-yl)-1-cyclohexanol, 4-(5,5,6-trimethylbicyclo[2.2.1]hept-2-yl)-1-cyclohexanol, 2-(1,7,7-trimethylbicyclo[2.2.1]hept-2-yl)-1-cyclohexanol, 3-methylindole, 8-mercapto-3-p-menthanone, 3,7-dimethyl-3-octanol, 1,3,3-trimethylbicyclo[2.2.1]heptan-2-one, 1-isopropyl-4-methylbicyclo [3.1.0] hexan-3-one, 1-isopropyl-4-methylbicyclo[3.1.0]hexan-3-one, thymol, 6-hexyltetrahydro-2H-pyran-2-one, 3-propylphenol, 2-cyclohexylethyl acetate, octanal, 1,3-nonanediyl diacetate, tetrahydro-3-pentyl-4(2H)-pyranyl acetate, 5-heptyldihydro-2(3H)-furanone, 3-butylidene-1-benzo[C] furanone, 3,7-dimethyl-6-octen-1-ol, 3,7-dimethyl-6-octenenitrile, coriander oil, cyclopropylmethyl (3Z)-3-hexenoate, cyclopropylmethyl (3E)-3-hexenoate, 3,7-dimethyl-1-octen-3-ol, cinnamon leaf oil, 4-decanolide, 3,5,6-trimethyl-3-cyclohexene-1-carbaldehyde, 2,4,6-trimethyl-3-cyclohexene-1-carbaldehyde, Lavandin Sumian essential oil, peppermint oil, 3,6,7-trimethyl-2,6-octadienal, petitgrain paraguay essential oil, (2Z)-4,8-dimethyl-2,7-nonadien-4-ol or a combination thereof

In a further embodiment of the invention the perfume ingredient is (4E,8E)-4,8-cyclododecadien-1-one, (4E,8Z)-4,8-cyclododecadien-1-one, (4Z,8E)-4,8-cyclododecadien-1-one, (4E)-4-methyl-5-(4-methylphenyl)-4-pentenal, 3,7-dimethyl-6-octenenitrile,: 3,7-dimethyl-6-octen-1-ol, (2Z)-4,8-dimethyl-2,7-nonadien-4-ol, 1,3-nonanediyl diacetate, tetrahydro-3-pentyl-4(2H)-pyranyl acetate, (Z)-4-decenal, 3-[4-methyl-3-cyclohexen-1-yl]-1-butanol, undecanal, (2E)-2-ethyl-4-[2,2,3-trimethyl-3-cyclopenten-1-yl]-2-buten-1-ol, 3,7-dimethyl-3-octanol, 2-(5,5,6-trimethylbicyclo[2.2.1]hept-2-yl)-1-cyclohexanol, 3-(5,5,6-trimethylbicyclo[2.2.1]hept-2-yl)-1-cyclohexanol, 4-(5,5,6-trimethylbicyclo[2.2.1]hept-2-yl)-1-cyclohexanol, 2-(1,7,7-trimethylbicyclo[2.2.1]hept-2-yl)-1-cyclohexanol, 2-cyclohexylethyl acetate, 1,3,3-trimethylbicyclo[2.2.1]heptan-2-one, 1-isopropyl-4-methylbicyclo[3.1.0]hexan-3-one or a combination thereof.

In an embodiment of the invention the perfume ingredient is cyclopropylmethyl (3Z)-3-hexenoate or cyclopropylmethyl (3E)-3-hexenoate, 3,6,7-trimethyl-2,6-octadienal, (2Z)-4,8-dimethyl-2,7-nonadien-4-ol, octanal, 5-heptyldihydro-2 (3H)-furanone, 3,7-dimethyl-6-octen-1-ol, 3,7-dimethyl-6-octenenitrile, 3,7-dimethyl-1-octen-3-ol, cinnamon leaf oil, Lavandin Sumian essential oil, 2-cyclohexylethyl acetate, peppermint oil, coriander oil, Petitgrain paraguay essential oil, 3-butylidene-1-benzo[C] furanone or a combination thereof.

A perfume ingredient may be at least 0.001% (w/v) of the antimicrobial composition. A perfume ingredient of the present invention may be from about 0.001% to about 5.0% w/v of the antimicrobial composition. Preferably, the perfume ingredient is from about 0.01% to about 5.0% w/v of the antimicrobial composition. Further preferably, the perfume ingredient is from about 0.05% to about 5.0% w/v of the antimicrobial composition. In further aspect, the perfume ingredient is from about 0.1% to about 5.0% w/v of the antimicrobial composition.

The concentration of a perfume ingredient for a bactericidal effect of 5.5 log reduction of the present invention may be less than or equal to 0.1% in the ethanol solution.

A “hard surface” as used herein refers to any hard surface. Surfaces to be cleaned include kitchens and bathrooms, e.g., floors, walls, tiles, windows, cupboards, sinks, showers, shower plastified curtains, wash basins, WCs, fixtures and fittings and the like made of different materials like ceramic, vinyl, no-wax vinyl, linoleum, melamine, glass, steel, kitchen work surfaces, any plastics, plastified wood, metal or any painted or varnished or sealed surface and the like. Household hard surfaces also include household appliances including, but not limited to refrigerators, freezers, washing machines, automatic dryers, ovens, microwave ovens, dishwashers and so on. Such hard surfaces may be found both in private households as well as in commercial, institutional and industrial environments.

A “body part” as used herein refers to any part of a mammalian body that is exposed to the external environment and includes skin and mucosal surfaces. Thus, for example, a body part includes skin, oral mucosa, and teeth. In a preferred embodiment, the body part is a human body part.

A surfactant according to the present invention may be, but is not limited to, selected from the group of anionic, amphoteric, non-ionic or cationic surfactants.

Non limiting examples of anionic surfactants include sodium, potassium, or ammonium salts of alkyl sulfonates, fatty acid methylester sulfonates, alkyl benzene sulfonates, secondary alkane sulfonate, alpha olefin sulfonates, alcohol sulfates, alcohol ether sulfates, alcohol ether phosphates, sulfated alkanolamides, glyceride sulfates, fatty acids, dialkylsulfosuccinates, N-acyl-sarcosinates, N-acyl-taurates, acyl-isethionates, N-acyl-glutamates, N-acyl-glycinates, and N-acyl-alaninates.

Non limiting examples of amphoteric surfactants include alkyl betaines, alkyl amidopropyl betaines, alkyl sulfobetaines, alkyl amine oxides, lecithin (phospholipids) such as phosphatidylcholine, lysolecithin, alkyl-amphoacetate, and alkyl-amphodiacetate.

Non limiting examples of non-ionic surfactants include ethoxylated aliphatic alcohols, ethoxylated alkyl phenols, ethoxylated thiols, mixed propoxylated and ethoxylated aliphatic alcohols, ethoxylated castor oil or hydrogenated castor oil, acid ethoxylated fatty acids, fatty esters of hexitols and cyclic anhydrohexitols (e.g., sorbitan), fatty esters of ethoxylated hexitols and cyclic anhydrohexitols (e.g. polysorbate), sugar esters, alkyl polyglycosides, polyglyceryl fatty acid esters, ethoxylated amines, ethoxylated amides, and alkyl diethanolamides.

Non-ionic surfactants can be selected from the group of water-soluble triblock copolymers comprising blocks of polyethyleneglycol and polypropyleneglycol (sold under trade names such as Pluronic, Tetronic, Poloxamer, Syperonics etc.).

Surfactants can also be chosen from the group of natural biosurfactants including glycolipids (e.g., sophorolipids, mannosylerythritollipids and rhamnolipids) and saponins.

Surfactants can also be chosen from the group of cationic surfactants including alkyl quaternary ammonium salts, esterquats, linear alkyl-amines, amide-amines, ester-amines, or ethoxylated amines.

Surfactants can be used as combinations of above mentioned surfactants.

In one embodiment the anionic surfactant is sodium lauryl ether sulfate.

In another embodiment the amphoteric surfactant is cocamidopropyl betaine.

In another embodiment the non-ionic surfactant is coco-glucoside.

A surfactant may be 0.1% to 30% (w/w) of the antimicrobial composition. In certain aspects, a surfactant of the present invention may be about 0.1% to about 20% w/w of the total weight of the antimicrobial composition. In further aspect, the surfactant is about 1% to about 10% w/w of the total weight of the antimicrobial composition.

A surfactant may be 0.1% to 30% (w/w) of the antimicrobial composition. In certain aspects, the surfactant is 0.1% to 20% (w/w) of the antimicrobial composition. In further aspects, the surfactant is 0.1% to 10% (w/w) of the antimicrobial composition. In a further aspect the surfactant is 9.1% to 30% (w/w) of the antimicrobial composition. In a further aspect the surfactant is 9.1% to 20% (w/w) of the antimicrobial composition. In a further aspect the surfactant is 9.1% to 10% (w/w) of the antimicrobial composition.

An antimicrobial composition of the present invention further comprises a hydrotrope. A hydrotrope is a substance in the presence of which the solubility of a hydrophobic compound in water is enhanced, while it does not form a microemulsion or lyotropic liquid crystals by itself.

A hydrotrope of the present invention may be about 0.5% to about 20% w/w of the total weight of the antimicrobial composition. In an aspect, the hydrotrope is about 1% to about 10% w/w of the total weight of the antimicrobial composition

In a preferred embodiment the antimicrobial composition of the present invention comprises less than 4% (w/w) hydrotrope, less than 3% (w/w) hydrotrope, less than 2% (w/w) hydrotrope, less than 1% (w/w) hydrotrope, or less.

Furthermore in another preferred embodiment, the antimicrobial composition comprises surfactant at more than or equal to 10% (w/w) of the antimicrobial composition then the hydrotrope is more than 4% (w/w) of the antimicrobial composition, preferably more than 5% (w/w) of the antimicrobial composition, preferably more than 6% (w/w) of the antimicrobial composition, preferably more than 7% (w/w) of the antimicrobial composition, preferably more than 10% (w/w) of the antimicrobial composition, preferably more than 15% (w/w) of the antimicrobial composition. Hydrotropes can be selected from the group of aryl sulfonates. In certain aspects the hydrotrope is benzene sulfonate, toluene sulfonate, xylene sulfonate, cumene sulfonate, or combinations thereof, and in the form of the corresponding sodium, ammonium or potassium salts.

The hydrotrope can also be selected from the group of diisobutyl sulfosuccinate, diisopropyl sulfosuccinate, di-n-propyl sulfosuccinate, diethyl sulfosuccinate, or combinations thereof, and in the form of the corresponding sodium, ammonium or potassium salts.

The hydrotrope can be selected from the group of benzoate, salicylate, or butyl monoglycol sulfate, and in the form of the corresponding sodium, ammonium or potassium salts.

The hydrotrope can be dipropyleneglycol-n-butyl ether.

The hydrotrope can be catechol, resorcinol, pyrogallol, hydroquinone, or 4-methoxyphenol.

The hydrotrope can be selected from the group of benzyl alcohol, urea, nicotinamide,

The hydrotrope can be sodium benzoate or sodium acetate.

The hydrotrope can be a short chain (ca. C4) alkyl polyglycoside.

The hydrotrope can be used as combinations of above mentioned hydrotropes.

Non-limiting examples of suitable hydrotropes include: toluene-sulfonate, xylene-sulfonate, cumene-sulfonate, diisobutyl-sulfosuccinate, sodium salicylate, sodium acetate and sodium benzoate. An antimicrobial composition of the present invention may further comprise a solvent. According to an embodiment, the antimicrobial composition comprises a water-miscible co-solvent, preferably chosen in the group consisting of mono- and polyhydric solvents. Non limiting examples of such solvents can be found from the group containing ethanol, n-propanol, propylene glycol, hexylene glycol, dipropylene glycol, glycerol, isopropylidene glycerol, butylene glycol (1,3-butanediol), 1,2-pentanediol, 1,2-hexanediol, 1,3-propanediol, and isopropanol, and mixtures thereof. According to another embodiment the water miscible co-solvent is chosen in the group of triethylcitrate, triacetin, ethyl lactate, glycol ethers.

An antimicrobial composition of the present invention may further comprise optional ingredients such as colorants, preservatives, viscosifiers, opacifiers, emollients, humectants, antioxidants, gelling agents, gums, chelators, functional polymers, cellulose derivatives, essential oils, electrolytes, and pH adjusters.

The present invention encompasses consumer products including the antimicrobial composition such as, for example, a personal cleaning product, an oral care product, a deodorant product, a hard surface cleaning product, liquid soap, foaming soap, liquid detergent, shampoo, shower gel, facial cleanser, mouthwash, and toothpaste.

An antimicrobial composition according to the present invention may be active against Gram-negative and Gram-positive bacteria. Further, an antimicrobial composition according to the present invention may be active against the following bacteria: Escherichia coli, Salmonella sp., Pseudomonas aeruginosa, Pseudomonas fluorescens, Serratia marcescens, Klebsiella pneumoniae, Staphylococcus aureus and Listeria monocytogenes or the combination thereof.

An antimicrobial composition according to the present invention may also be active against fungal species, particularly Aspergillus, Penicillium, Cladosporium, and Candida species of fungi.

As can be appreciated by the skilled person, bacterial and fungal species listed above can contribute to unwanted personal odors and odors on clothes and in the home. In particular Moraxella and Pseudomonas species of bacteria, and Cladosporium species of fungi are well known to cause dampness or moldy smells on cloth and in laundry.

Hence a preferred embodiment of the invention is wherein the consumer products including the antimicrobial composition of the invention is a cleaning product, preferably a laundry product, and reduces undesirable dampness or moldy odors.

A further aspect of the invention provides a method for eliminating or/and reducing moldy odor from clothes comprising contacting the clothes with the antimicrobial composition of the invention.

### Malodor Antagonists

An antimicrobial composition of the present invention may be used in combination (e.g., in one composition, or in separate compositions that are administered simultaneously or near in time) with a malodor antagonist system. A malodorous compound may activate at least one olfactory receptor associated with the malodor. Without intending to be limited to any particular theory, malodors are usually complex mixtures of more than one malodorous compound which may include various amines, thiols, sulfides, short chain aliphatic and unsaturated acids, e.g. fatty acids, and their derivatives. In one aspect, the at least one olfactory receptor is an olfactory receptor disclosed in International Patent Application Publication No. WO2019/101821 A1. In an alternate aspect, the at least one olfactory receptor is an olfactory receptor disclosed in International Patent Application Publication No. WO2018/091686 A1. In an alternate aspect, the at least one olfactory receptor is an olfactory receptor disclosed in International Patent Application Publication No. WO2018/091686 A1.

In one aspect, the inhibition of the at least one olfactory receptor inhibits, reduces, suppresses, the perception of a malodor in a consumer.

As used herein, the terms “antagonists,” “inhibitor,” “blockers,” “suppressors,” “counteractants” and “modulators” of olfactory receptors are used interchangeably to refer to inhibitory, blocking, suppressing, or modulating molecules identified using in vivo, ex vivo and in vitro assays for olfactory transduction, e.g., ligands, antagonists, and their homologs and mimetics. Inhibitors are compounds that, e.g., bind to, partially or totally block stimulation, decrease, suppress, prevent, delay activation, inactivate, desensitize, or down regulate olfactory transduction, e.g., antagonists. Activators are compounds that, e.g., bind to, stimulate, increase, open activate, facilitate, enhance activation, sensitize, or up regulate olfactory transduction, e.g., agonists. Modulators include compounds that, e.g., alter the interaction of a receptor with: extracellular proteins that bind activators or inhibitor (e.g., odourant-binding proteins, ebnerin and other members of the hydrophobic carrier family); G proteins; kinases (e.g., homologs of rhodopsin kinase and beta adrenergic receptor kinases that are involved in deactivation and desensitization of a receptor); and arrestins, which also deactivate and desensitize receptors.

The ability of compounds and methods of the present disclosure to inhibit or antagonize the at least one olfactory receptor may be determined by any suitable method readily selected by one of ordinary skill in the art, such as, for example, via an ex vivo cultured neuron assay, or via an in vitro assay using a cell line that expresses a butyric acid olfactory receptor.

As used herein, the term “olfactory receptor”, or “OR” refers to one or more members of a family of G protein-coupled receptors (GPCRs) that are expressed in olfactory cells. Olfactory receptor cells can also be identified on the basis of morphology or by the expression of proteins specifically expressed in olfactory cells. OR family members may have the ability to act as receptors for odorants and induce an olfactory transduction cascade.

In one aspect, the at least one compound that inhibits the activity of at least one olfactory receptor is selected from the group consisting of: benzyl acetate, isobornyl acetate, undec-10-enal, undec-9-enal, Virginia cedarwood essential oil, 3,7-dimethyl-2,6-octadienal, 3,7-Dimethyl-6-octen-1-ol, 3,7-dimethyloct-6-enenitrile, coumarin, (2E)-1-(2,2-dimethyl-6-methylenecyclohexyl)-2-buten-1-one, methyl ionone gamma, (Z)-3,4,5,6,6-pentamethylhept-3-en-2-one, 2,6-dimethylhept-5-enal, menthone, 1-(5,5-dimethyl-1-cyclohexenyl) pent-4-en-1-one, patchouli essential oil, 2,6-nonadienal, (2-tert-butylcyclohexyl) acetate, vertoxime, and 2-Methoxynaphthalene.

Examples of other compounds capable of inhibiting the activity of at least one olfactory receptor selected from the group consisting of: a DMTS olfactory receptor, an indole/skatole olfactory receptor, a butyric acid olfactory receptor, and a p-cresol olfactory receptor include the compounds disclosed in International Patent Application Publication No. WO2019/101821 A1.

Additional examples of other compounds capable of inhibiting the activity of at least one olfactory receptor selected from the group consisting of: a DMTS olfactory receptor, an indole/skatole olfactory receptor, a butyric acid olfactory receptor, and a p-cresol olfactory receptor include the compounds disclosed in International Patent Application Publication No. WO2018/091686 A1.

In one aspect, an at least one compound capable of inhibiting the activity of a DMTS olfactory receptor may be selected from the compounds capable of inhibiting the activity of a DMTS olfactory receptor disclosed in International Patent Application Publication No. WO2019/101821 A1.

In one aspect, an at least one compound capable of inhibiting the activity of a butyric acid olfactory receptor may be selected from the compounds capable of inhibiting the activity of a butyric acid olfactory receptor disclosed in International Patent Application Publication No. WO2019/101821 A1.

In one aspect, an at least one compound capable of inhibiting the activity of an indole/skatole olfactory receptor may be selected from the compounds capable of inhibiting the activity of an indole/skatole olfactory receptor disclosed in International Patent Application Publication No. WO2019/101821 A1.

In one aspect, an at least one compound capable of inhibiting the activity of a p-cresol olfactory receptor may be selected from the compounds capable of inhibiting the activity of a p-cresol olfactory receptor disclosed in International Patent Application Publication No. WO2018/091686 A1.

In one aspect, the malodor antagonist system is present in the antimicrobial composition in an amount from 30 to 50% wt%, relative to the antimicrobial composition.

In one aspect, the malodor antagonist system is present in the antimicrobial composition in an amount from 30 to 45, or alternatively, in an amount from 30 to 40, or alternatively, in an amount from 30 to 35 wt %, relative to the antimicrobial composition.

In one aspect, the malodor antagonist system is present in the antimicrobial composition in an amount from 35 to 50, or alternatively, from 40 to 50, or alternatively, from 45 to 50 wt %, relative to the antimicrobial composition.

In one aspect, the malodor antagonist system is present in the antimicrobial composition at 30, or 35, or 40, or 45, or 50 wt %, relative to the antimicrobial composition.

### Malodor Neutralizing System

An antimicrobial composition of the present invention may be used in combination (e.g., in one composition, or in separate compositions that are administered simultaneously or near in time) with a malodor neutralizing system. A malodor neutralizing system limits, decreases or eliminates the perception of a malodor by reacting with various chemical compounds that may be responsible for the malodor. The reactions result in reduction of the malodor material's airborne levels and consequent reduction in the perception of the malodor.

In one aspect, the at least one malodor neutralizing system is selected from the group consisting of: a) a composition comprising at least one ingredient selected from the group consisting of: (i) at least one aldehyde of formula R¹CHO, wherein R¹ is an aliphatic linear or branched, saturated or unsaturated carbon chain containing from 1 to 12 carbon atoms; (ii) at least one ketone of formula R²COR³, wherein R² is an ethyl or methyl group and R³ is an aliphatic linear or branched, saturated or unsaturated carbon chain containing from 1 to 12 carbon atoms; and (iii) primary alcohols of formula R⁴CH₂OH, wherein R⁴ is an aliphatic linear or branched, saturated or unsaturated carbon chain containing 1 to 12 carbon atoms, optionally substituted with an aromatic moiety; b) a composition comprising: (i) at least one ingredient selected from the group consisting of (2E)-1-(2,6,6-trimethyl-2-cyclohexen-1-yl)-2-buten-1-one, (2E)-1-(2,6,6-trimethyl-1-cyclohexen-1-yl)-2-buten-1-one, (2E)-1-(2,2-dimethyl-6-methylenecyclohexyl)-2-buten-1-one, (E)-1-(2,6,6-trimethyl-3-cyclohexen-1-yl)-2-buten-1-one, 1-(5,5-dimethyl-1-cyclohexen-1-yl)-4-penten-1-one, (+−)-methyl-2,2-dimethyl-6-methylene-1-cyclohexanecarboxylate, α- or β-(E)-4-(2,6,6-trimethyl-2-cyclohexen-1-yl)-3-buten-2-one (α- or β-ionone), (1E)-1-(2,6,6-trimethyl-2-cyclohexen-1-yl)-1-penten-3-one, (1E)-1-(2,6,6-trimethyl-1-cyclohexen-1-yl)-1-penten-3-one, (E)-3-methyl-4-(2,6,6-trimethyl-2-cyclohexen-1-yl)-3-buten-2-one, 1-(2,6,6-trimethyl-1(2)-cyclohexen-1-yl)-1,6-heptadien-3-one and 1-(4,6,6-trimethyl-1,3-cyclohexadien-1-yl)-2-buten-1-one; and (ii) at least one nitrile ingredient selected from the group consisting of 3-phenyl-2-propenenitrile, (E/Z)-3-methyl-5-phenyl-2-pentenenitrile, 3,7-dimethyl-6-octenenitrile, 2-propyl-1-heptanenitrile, dodecanenitrile, and the mixture of 3-(2,3-dimethyl-2(3)-cyclopenten-1-yl) butanenitrile and 3-(2-methyl-3-methylene-1-cyclopentyl)butanenitrile; and c) a combination of a) and b).

Examples of compositions comprising at least one ingredient selected from the group consisting of: (i) at least one aldehyde of formula R¹CHO, wherein R¹ is an aliphatic linear or branched, saturated or unsaturated carbon chain containing from 1 to 12 carbon atoms; (ii) at least one ketone of formula R²COR₃, wherein R² is an ethyl or methyl group and R³ is an aliphatic linear or branched, saturated or unsaturated carbon chain containing from 1 to 12 carbon atoms; and (iii) primary alcohols of formula R⁴CH₂OH, wherein R⁴ is an aliphatic linear or branched, saturated or unsaturated carbon chain containing 1 to 12 carbon atoms, optionally substituted with an aromatic moiety may be found in U.S. Pat. No. 8,772,354.

Examples of compositions comprising at least one ingredient selected from the group consisting of (i) at least one ingredient selected from the group consisting of (2E)-1-(2,6,6-trimethyl-2-cyclohexen-1-yl)-2-buten-1-one, (2E)-1-(2,6,6-trimethyl-1-cyclohexen-1-yl)-2-buten-1-one, (2E)-1-(2,2-dimethyl-6-methylenecyclohexyl)-2-buten-1-one, (E)-1-(2,6,6-trimethyl-3-cyclohexen-1-yl)-2-buten-1-one, 1-(5,5-dimethyl-1-cyclohexen-1-yl)-4-penten-1-one, (+−)-methyl-2,2-dimethyl-6-methylene-1-cyclohexanecarboxylate, α- or β-(E)-4-(2,6,6-trimethyl-2-cyclohexen-1-yl)-3-buten-2-one (α- or β-ionone), (1E)-1-(2,6,6-trimethyl-2-cyclohexen-1-yl)-1-penten-3-one, (1E)-1-(2,6,6-trimethyl-1-cyclohexen-1-yl)-1-penten-3-one, (E)-3-methyl-4-(2,6,6-trimethyl-2-cyclohexen-1-yl)-3-buten-2-one, 1-(2,6,6-trimethyl-1(2)-cyclohexen-1-yl)-1,6-heptadien-3-one and 1-(4,6,6-trimethyl-1,3-cyclohexadien-1-yl)-2-buten-1-one; and (ii) at least one nitrile ingredient selected from the group consisting of 3-phenyl-2-propenenitrile, citronitrile, citronellyl nitrile, 2-propyl-1-heptanenitrile, dodecanenitrile, and the mixture of 3-(2,3-dimethyl-2(3)-cyclopenten-1-yl)butanenitrile and 3-(2-methyl-3-methylene-1-cyclopentyl)butanenitrile may be found in U.S. Patent Application Publication No. 2017/0266334 A1.

In one aspect, the at least one malodor neutralizing system is present in the antimicrobial composition in an amount from 5 to 20% wt%, relative to the antimicrobial composition.

In one aspect, the at least one malodor neutralizing system is present in the antimicrobial composition in an amount from 5 to 19, or alternatively, in an amount from 5 to 18, or alternatively, in an amount from 5 to 17, or alternatively, in an amount from 5 to 16, or alternatively, in an amount from 5 to 15, or alternatively, in an amount from 5 to 14, or alternatively, in an amount from 5 to 13, or alternatively, in an amount from 5 to 12, or alternatively, in an amount from 5 to 11, or alternatively, in an amount from 5 to 10, or alternatively, in an amount from 5 to 9, or alternatively, in an amount from 5 to 8, or alternatively, in an amount from 5 to 7, or alternatively, in an amount from 5 to 6 wt %, relative to the antimicrobial composition.

In one aspect, the at least one malodor neutralizing system is present in the antimicrobial composition in an amount from 6 to 20, or alternatively, in an amount from 7 to 20, or alternatively, in an amount from 8 to 20, or alternatively, in an amount from 9 to 20, or alternatively, in an amount from 10 to 20, or alternatively, in an amount from 11 to 20, or alternatively, in an amount from 12 to 20, or alternatively, in an amount from 13 to 20, or alternatively, in an amount from 14 to 20, or alternatively, in an amount from 15 to 20, or alternatively, in an amount from 16 to 20, or alternatively, in an amount from 17 to 20, or alternatively, in an amount from 18 to 20, or alternatively, in an amount from 19 to 20 wt %, relative to the antimicrobial composition.

In one aspect, the at least one malodor neutralizing system is present in the malodor counteracting composition at 5, or 6, or 7, or 8, or 9, or 10, or 11, or 12, or 13, or 14, or 15, or 16, or 17, or 18, or 19, or 20 wt %, relative to the antimicrobial composition.

The present invention is illustrated by, but is not limited to, the following examples.

### EXAMPLES

### Example 1: Determination of Antimicrobial Efficacy of Fragrance Materials in Ethanol Solution

a. Preparation of Bacterial Suspensions

Bacterial suspensions of E. coli ATCC 10536 were prepared for antimicrobial testing as follows. Stock cultures stored at −80° C. were sub-cultured onto Tryptic Soy Agar (TSA) plate, and incubated at 37° C. for 24 h to obtain single colonies. Single colonies of the primary cultures were streaked onto TSA plates and incubated at 37° C. for 24 h to prepare the secondary cultures. Single colonies of secondary cultures were inoculated into 50 ml of Tryptic Soy broth (TSB), incubated at 37° C. 180 rpm for 18 h. Aliquots (0.5 ml) of the 18 h culture were inoculated into 50 ml of fresh TSB, and incubated at 37° C. 180 rpm for 2-3 hours. When the OD600 nm value of the broth reached 1-2, cells were harvested by centrifugation at 5,000 rpm for 10 min, and then resuspended in the same fresh broth media to achieve the target level of 1-5×10⁸ colony forming unit (CFU)/mL. This suspension was used for further antimicrobial tests.

b. Determination of the Bactericidal Activity of Perfume Ingredients in Ethanol Solutions

Unless indicated otherwise, log reduction testing according to the present invention is determined in accordance with the following method.

Bacterial contact time (BCT) test based on European standard EN-1276 was used to determine dose dependent bactericidal activity of perfume ingredients in 20% ethanol solutions.

Perfume ingredients of various concentrations (doses) were prepared in 40% ethanol solutions. Eleven replicates of each sample were added into 96-well microtiter plates (120 μl per well) in column 1-11 of one row, with row B as the control sample of the ethanol solution. Then, 120 μl of cell suspension at a concentration of approximately 1-5×10⁸ CFU/ML (as prepared above) were added to each well of the microtiter plate. A specified contact time (45 s) was allowed for target bacterial strains. At the end of the contact time, serial dilutions were prepared in 96 well plates with growth media: 3 times of 1 in 10 dilutions and followed by 17 times of 1 in 2 dilutions. Each plate was sealed and incubated at 37° C. under agitation (180 rpm). After incubation, turbidity of the wells (OD 600 nm) was recorded by Tecan microplate reader. Turbid cells were regarded as positive growth of viable cells. The total number of viable cells (log CFU/mL) for each sample was calculated. And the log reduction for each test composition at the final concentration (dose) was calculated against the control sample of the 20% ethanol solution.

### Example 2: Determination of the Antimicrobial Efficacy of Perfume Ingredients in Different Surfactant Bases

a. Preparation of Test Samples

Test samples were prepared by mixing perfume ingredients with the surfactant bases and vigorously stirred for 24 hrs. Samples with transparent appearance were selected for antimicrobial testing.

b. Measurement of Antimicrobial Efficacy of Fragrance Materials in Surfactant Systems

Antimicrobial efficacy was tested against a representative Gram-negative bacterial strain, Escherichia coli ATCC 10536, using robotic bacteria contact time (BCT) test based on European standard EN-1276 and EP2787827 (B1).

Preparation of screening plate and dilution plate: Aliquots (270 μl) of compositions were dispensed into the wells of a 96-well microtiter plate (MTP) along two columns (B1-H1, and B7-H7), and 270 μl of MilliQ water was added to well A1 and A7 as the control samples. This MTP was labelled as the “Screening plate”. In another MTP, labelled as the “Dilution plate”, 270 μL of Dey-Engley (D/E) neutralizing solution was added to column 1 and column 7. 270 μl of tryptone diluent solution was added to columns 2-6 and columns 8-12 of the Dilution MTP by a Hamilton robotic liquid handling station.

BCT test & neutralization & dilution: Bacterial stock (30 μl) was then added to columns 1 of the ‘Screening plate’ and mixed by a Hamilton robotic liquid handling station. After a contact time of 45 seconds, 30 μl of the mixtures in column 1 were transferred into the corresponding wells of column 1 of the ‘Dilution plate’. After neutralizing for 5 minutes in the D/E neutralizing solution, 30 μl of the neutralized mixtures were transferred from column 1 to column 2 of the Dilution MTP and mixed, followed by transferring 30μl of the mixtures from column 2 into column 3. This process was repeated serially diluting the bacteria suspensions across the plate to column 6. Bacterial stock (30 μl) was then added to columns 7 of the ‘Screening plate’ and mixed by a Hamilton robotic liquid handling station. After a contact time of 45 seconds, 30 μl of the mixtures in column 7 were transferred into the corresponding wells of column 7 of the ‘Dilution plate’. After neutralizing for 5 minutes in the D/E neutralizing solution, 30 μl of the mixtures were transferred from column 7 to column 8 of the Dilution MTP and mixed, followed by transferring further 30μl mixtures from column 8 into column 9. This process was repeated serially diluting the bacteria suspensions across the plate to column 12.

Plating: 30 μl volumes from each well in the Dilution MTP were transferred onto four Tryptone Soya Agar (TSA) plates. The TSA plates were allowed to stand for about two hours so that the 30 μl inocula spots could dry and the plates were then inverted and incubated overnight at 37° C. for 24 hours. After incubation, colonies were counted.

Calculating log reduction: Select the dilutions with colony counts, and calculate the viable cell counts (CFU/mL) of mixture in ‘Screening plate’, and log reduction against the control MilliQ sample was calculated.

### Results

Perfume ingredients with high bactericidal activity in ethanol solutions: at least a 5.5 log reduction in bacterial viability at less than or equal to 0.1% (w/v) of the bactericidal perfume ingredients in ethanol solution against at least one Gram-negative bacterium, preferably at least one of Escherichia coli, Salmonella sp., Pseudomonas aeruginosa, Pseudomonas fluorescens, Serratia marcescens, and Klebsiella pneumoniae, when tested according to the test procedure as described in Example 1.

Perfume ingredient with high bactericidal activity: 1-phenylethyl acetate, 1-octanol, (1,3,3-trimethylbicyclo[2.2.1]heptan-2-ol, undecanal, (2E)-2-methyl-3-phenyl-2-propenal, (Z)-2-nonenal, 1-(5-propyl-1,3-benzodioxol-2-yl)ethenone, (2E,6Z)-2,6-nonadien-1-ol, ethyl benzoate, 1,7,7-trimethylbicyclo[2.2.1]heptan-2-ol, benzyl butanoate, 1-butoxycarbonylethyl butanoate, ethyl hexanoate, 5-isopropyl-2-methylphenol, (4E,8E)-4,8-cyclododecadien-1-one, (4E,8Z)-4,8-cyclododecadien-1-one, (4Z,8E)-4,8-cyclododecadien-1-one, methyl (2E)-2-methyl-2-hexenoate, ethyl (E)-3-phenyl-2-propenoate, methyl (E)-3-phenyl-2-propenoate, (Z)-3,7-dimethyl-2,6-octadienal, (E)-3,7-dimethyl-2,6-octadienal, 3,5,5-trimethyl-1-hexanol, 3-[4-methyl-3-cyclohexen-1-yl]-1-butanol, (2E)-2-ethyl-4-[2,2,3-trimethyl-3-cyclopenten-1-yl]-2-buten-1-ol, 4-decanolide, (Z)-4-decenal, 2-methoxy-4-propylphenol, 2,6-dimethyl-7-octen-4-one, 2,6-dimethyl-4-heptanol, (E)-3,7-dimethyl-2,6-octadien-1-ol, 2,4,6-trimethyl-3-cyclohexene-1-methanol, 2-methoxy-4-[(1E)-1-propen-1-yl]phenol, (2E)-2-methyl-3-(4-methylphenyl)-2-propen-1-ol, 2,5-dimethyl-2-indanmethanol, 1,2-dimethoxy-4-[(1Z)-1-propen-1-yl]benzene, 1,2-dimethoxy-4-[(1E)-1-propen-1-yl]benzene, (4E)-4-methyl-5-(4-methylphenyl)-4-pentenal, (2,2-dimethyl-3-[(2Z)-3-methyl-2,4-pentadien-1-yl]oxirane, (2,2-dimethyl-3-[(2E)-3-methyl-2,4-pentadien-1-yl]oxirane, 2-ethyl-1-hexanol, (Z)-3,7-dimethyl-2,6-octadien-1-ol, 1-(3-methyl-1-benzofuran-2-yl)ethenone, (Z)-6-nonen-1-ol, (2E,6Z)-2,6-nonadienal, 1,8-p-menthadien-7-ol, 3-methyl-5-phenyl-1-pentanol, 2,6,6-trimethyl-1,3-cyclohexadiene-1-carbaldehyde, ethyl 2-hydroxybenzoate, 2-(5,5,6-trimethylbicyclo[2.2.1]hept-2-yl)-1-cyclohexanol, 3-(5,5,6-trimethylbicyclo[2.2.1]hept-2-yl)-1-cyclohexanol, 4-(5,5,6-trimethylbicyclo[2.2.1]hept-2-yl)-1-cyclohexanol, 2-(1,7,7-trimethylbicyclo[2.2.1]hept-2-yl)-1-cyclohexanol, 3-methylindole, 8-mercapto-3-p-menthanone, 3,7-dimethyl-3-octanol, 1,3,3-trimethylbicyclo[2.2.1]heptan-2-one, 1-isopropyl-4-methylbicyclo[3.1.0]hexan-3-one, 1-isopropyl-4-methylbicyclo[3.1.0]hexan-3-one, thymol, 6-hexyltetrahydro-2H-pyran-2-one, 3-propylphenol, 2-cyclohexylethyl acetate, octanal, 1,3-nonanediyl diacetate, tetrahydro-3-pentyl-4(2H)-pyranyl acetate, 5-heptyldihydro-2(3H)-furanone, 3-butylidene-1-benzo[C] furanone, 3,7-dimethyl-6-octen-1-ol, 3,7-dimethyl-6-octenenitrile, coriander oil, cyclopropylmethyl (3Z)-3-hexenoate, cyclopropylmethyl (3E)-3-hexenoate, 3,7-dimethyl-1-octen-3-ol, cinnamon leaf oil, 4-decanolide, 3,5,6-trimethyl-3-cyclohexene-1-carbaldehyde, 2,4,6-trimethyl-3-cyclohexene-1-carbaldehyde, Lavandin Sumian essential oil, peppermint oil, 3,6,7-trimethyl-2,6-octadienal, petitgrain paraguay essential oil, (2Z)-4,8-dimethyl-2,7-nonadien-4-ol or a combination thereof.

TABLE 1
Surfactant base compositions (pH 5.5-6.0)

| | A | B |
|---|---|---|
| | Concentration | Concentration |
| | (% wt) | (% wt) |
| Sodium laureth sulfate | 1.875 | 4.50 |
| (Texapon ® N70 ex. BASF) | | |
| Cocamidopropyl Betaine (Tego ® | 0.625 | 1.50 |
| Betain F50 ex. Evonik) | | |
| Citric acid | — | 0.07 |
| Sodium citrate | — | 0.42 |
| Water | Q.S. | Q.S. |

TABLE 2
Antimicrobial activity of selected perfume
ingredients in surfactant base A

| | | Concentration | Log reduction |
|---|---|---|---|
| | Perfume ingredients | (% wt) | (CFU/mL) |
| 1 | 5-heptyldihydro-2(3H)-furanone | 0.5 | 1.3 |
| 2 | 3,7-dimethyl-6-octen-1-ol | 0.5 | 2.8 |

TABLE 3
Effect of sodium cumene sulfonate (SCS) on the antimicrobial activity
of perfume ingredients (1% wt) in the surfactant base B

| | | SCS | Log reduction |
|---|---|---|---|
| | Perfume ingredients | (% wt) | (CFU/mL) |
| 3 | 2-cyclohexylethyl acetate | / | 0.0 |
| 4 | 2-cyclohexylethyl acetate | 3.00 | >5 |
| 5 | Octanal | / | Insoluble |
| 6 | Octanal | 3.00 | Insoluble |
| 7 | 5-heptyldihydro-2(3H)-furanone | / | −0.2 |
| 8 | 5-heptyldihydro-2(3H)-furanone | 3.00 | 4.9 |
| 9 | 3-butylidene-1-benzo[C] furanone | / | 0.2 |
| 10 | 3-butylidene-1-benzo[C] furanone | 3.00 | >5 |
| 11 | 3,7-dimethyl-6-octen-1-ol | / | Insoluble |
| 12 | 3,7-dimethyl-6-octen-1-ol | 3.00 | Insoluble |
| 13 | 3,7-dimethyl-6-octenenitrile | / | 0.1 |
| 14 | 3,7-dimethyl-6-octenenitrile | 3.00 | >5 |
| 15 | Coriander oil | / | −0.3 |
| 16 | Coriander oil | 3.00 | 4.3 |
| 17 | cyclopropylmethyl (3Z)-3-hexenoate OR | / | 0.1 |
| | cyclopropylmethyl (3E)-3-hexenoate | | |
| 18 | cyclopropylmethyl (3Z)-3-hexenoate OR | 3.00 | >5 |
| | cyclopropylmethyl (3E)-3-hexenoate | | |
| 19 | 3,7-dimethyl-1-octen-3-ol | / | −0.1 |
| 20 | 3,7-dimethyl-1-octen-3-ol | 3.00 | >5 |
| 21 | Cinnamon leaf oil | / | Insoluble |
| 22 | Cinnamon leaf oil | 3.00 | Insoluble |
| 23 | Lavandin Sumian essential oil | / | 0.0 |
| 24 | Lavandin Sumian essential oil | 3.00 | 4.4 |
| 25 | Peppermint oil | / | −0.1 |
| 26 | Peppermint oil | 3.00 | >5 |
| 27 | 3,6,7-trimethyl-2,6-octadienal | / | 3.3 |
| 28 | 3,6,7-trimethyl-2,6-octadienal | 3.00 | >5 |
| 29 | Petitgrain paraguay essential oil | / | 0.1 |
| 30 | Petitgrain paraguay essential oil | 3.00 | 3.1 |
| 31 | (2Z)-4,8-dimethyl-2,7-nonadien-4-ol | / | −0.1 |
| 32 | (2Z)-4,8-dimethyl-2,7-nonadien-4-ol | 3.00 | >5 |

TABLE 4
Effect of sodium salicylate on the antimicrobial activity
of perfume ingredients (1% wt) in the surfactant base B

| | | Sodium | Log reduction |
|---|---|---|---|
| | Perfume ingredients | salicylate | (CFU/mL) |
| 33 | 2-cyclohexylethyl acetate | 3.00 | >5 |
| 34 | Octanal | 3.00 | Insoluble |
| 35 | 5-heptyldihydro-2(3H)-furanone | 3.00 | >5 |
| 36 | 3-butylidene-1-benzo[C] furanone | 3.00 | Insoluble |
| 37 | 3,7-dimethyl-6-octen-1-ol | 3.00 | Insoluble |
| 38 | 3,7-dimethyl-6-octenenitrile | 3.00 | >5 |
| 39 | Coriander oil | 3.00 | >5 |
| 40 | cyclopropylmethyl (3Z)-3-hexenoate | 3.00 | >5 |
| | OR cyclopropylmethyl (3E)-3- | | |
| | hexenoate | | |
| 41 | 3,7-dimethyl-1-octen-3-ol | 3.00 | >5 |
| 42 | Cinnamon leaf oil | 3.00 | Insoluble |
| 43 | Lavandin Sumian essential oil | 3.00 | Insoluble |
| 44 | Peppermint oil | 3.00 | Insoluble |
| 45 | 3,6,7-trimethyl-2,6-octadienal | 3.00 | Insoluble |
| 46 | Petitgrain paraguay essential oil | 3.00 | Insoluble |
| 47 | (2Z)-4,8-dimethyl-2,7-nonadien-4-ol | 3.00 | >5 |

### Conclusions

In surfactant bases with low surfactant content (e.g., 2.5% of surfactant as shown in Table 1), perfume ingredients had high bactericidal activity. With the increasing amount of surfactant in the bases (e.g., 6% of surfactant as shown in Table 2), the antimicrobial activity of the perfume ingredients was dramatically decreased. As shown in Table 2 and 3, hydrotropes such as SCS or sodium salicylate could enhance the antimicrobial activity of perfume ingredients in surfactant bases. Not like in WO2006053458 A1, the hydrotrope amount in present invention can be lower than 4% to achieve high bactericidal activity in the surfactant bases.

### Example 3 In Vitro Efficacy of a Composition of the Invention in a Surface Cleaner Product

The anti-bac efficacy of a composition of the invention in a commercial surface cleaner sample, Pine-Sol® Multi-Surface Cleaner (Lemon Fresh) were determined. In brief, compositions of the invention were added to Pine-Sol® Multi-Surface Cleaner to final concentrations of 0.25%, 0.5%, 0.75%, 1.0% and 1.25%. Aliquots (120 μL) of each sample were mixed with equal amount of bacterial suspensions of E. coli ATCC 10536 as previously described in wells of 96 well plates, 11 replicates. After contact time of 3 min, viable cells (log CFU) of each well were enumerated. Bactericidal effect, measured as log reduction against control sample of MilliQ water.,

High antimicrobial efficacy was detected when adding 0.5-2% of compositions of the invention in the bases.

### Example 4 In Vitro Efficacy of Compositions of the Invention Presented Herein in a Soap Bar Base

Table 5 shows the compositions of soap bar base.

TABLE 5
Compositions of a soap bar base.

| Chemical name | INCI Nomenclature | CAS No. | % |
|---|---|---|---|
| Sodium salt of palm oil | Sodium Palmate | 61790-79-2 | 68.6 |
| fatty acid | | | |
| Sodium salt of palm | Sodium Palm Kernelate | 61789-89-7 | 17.15 |
| kernel oil fatty acid | | | |
| EDTA | Tetrasodium EDTA | 64-02-8 | 0.15 |
| HEDP | Tetrasodium etidronate | 3794-83-0 | 0.10 |
| Sodium chloride | Sodium chloride | 7647-14-5 | 0.5 |
| Glycerol | Glycerol | 56-8′1-5 | 0.5 |
| Water | Aqua | 7732-18-5 | 13 |

High antimicrobial efficacy was detected when adding 0.5-2% of compositions of the invention in the bases.

### Example 5 In Vitro Efficacy of Compositions of the Invention Presented Herein in a Roll-On Deodorant Soap Base

Table 6 shows the compositions of Roll-on deodorant soap base.

TABLE 6
Compositions of Roll-on deodorant soap base

| Composition | % |
|---|---|
| Water deionised | 50 |
| 2 NATROSOL 250 H Hydroxyethylcellulose | 0.7 |
| Ethyl alcohol 95° | 40 |
| 1,2-Propylene glycol | 5 |

High antimicrobial efficacy was detected when adding 0.5-1% of compositions of the invention in the bases.

1. An antimicrobial composition comprising:
a. a perfume ingredient having a bactericidal effect of a 5.5 log reduction in an ethanol solution at a concentration of less than or equal to 0.1% in ethanol solution;
b. a surfactant; and
c. a hydrotrope.

2. The antimicrobial composition of claim 1, wherein the perfume ingredient is at least 0.001% (w/v) of the antimicrobial composition.

3. The antimicrobial composition of claim 1, wherein the surfactant is selected from the group consisting of: an anionic surfactant, a non-ionic surfactant, an amphoteric surfactant, and a combination thereof.

4. The antimicrobial composition of claim 1, wherein the anionic surfactant is sodium lauryl ether sulfate, the amphoteric surfactant is cocamidopropyl betaine, and the non-ionic surfactant is coco-glucoside.

5. The antimicrobial composition of claim 1, wherein the antimicrobial composition comprises less than 4% hydrotrope.

6. The antimicrobial composition according to claim 5, wherein the hydrotrope is selected from the group consisting of: short chain alkyl polyglycosides, toluene sulfonate, xylene sulfonate, cumene sulfonate, diisobutyl sulfosuccinate, sodium salicylate, sodium acetate, and a combination thereof; or wherein the hydrotrope is a sodium, ammonium or potassium salt of a hydrotrope selected from the group consisting of: toluene sulfonate, xylene sulfonate, cumene sulfonate, diisobutyl sulfosuccinate; dipropyleneglycol n-butyl ether;
and a combination thereof.

7. The antimicrobial composition of claim 1, wherein the antimicrobial composition further comprises an additional agent active against gram-positive or gram-negative bacteria.

8. The antimicrobial composition of claim 1, wherein the antimicrobial composition further comprises a chelating agent selected from the group consisting of EDTA, and CDTA, and a combination thereof.

9. The antimicrobial composition of claim 1, wherein the perfume ingredient is selected from the group consisting of: 1-phenylethyl acetate, 1-octanol, (1,3,3-trimethylbicyclo[2.2.1]heptan-2-ol, undecanal, (2E)-2-methyl-3-phenyl-2-propenal, (Z)-2-nonenal, 1-(5-propyl-1,3-benzodioxol-2-yl)ethenone, (2E,6Z)-2,6-nonadien-1-ol, ethyl benzoate, 1,7,7-trimethylbicyclo[2.2.1]heptan-2-ol, benzyl butanoate, 1-butoxycarbonylethyl butanoate, ethyl hexanoate, 5-isopropyl-2-methylphenol, (4E,8E)-4,8-cyclododecadien-1-one, (4E,8Z)-4,8-cyclododecadien-1-one, (4Z,8E)-4,8-cyclododecadien-1-one, methyl (2E)-2-methyl-2-hexenoate, ethyl (E)-3-phenyl-2-propenoate, methyl (E)-3-phenyl-2-propenoate, (Z)-3,7-dimethyl-2,6-octadienal, (E)-3,7-dimethyl-2,6-octadienal, 3,5,5-trimethyl-1-hexanol, 3-[4-methyl-3-cyclohexen-1-yl]-1-butanol, (2E)-2-ethyl-4-[2,2,3-trimethyl-3-cyclopenten-1-yl]-2-buten-1-ol, 4-decanolide, (Z)-4-decenal, 2-methoxy-4-propylphenol, 2,6-dimethyl-7-octen-4-one, 2,6-dimethyl-4-heptanol, (E)-3,7-dimethyl-2,6-octadien-1-ol, 2,4,6-trimethyl-3-cyclohexene-1-methanol, 2-methoxy-4-[(1E)-1-propen-1-yl]phenol, (2E)-2-methyl-3-(4-methylphenyl)-2-propen-1-ol, 2,5-dimethyl-2-indanmethanol, 1,2-dimethoxy-4-[(1Z)-1-propen-1-yl]benzene, 1,2-dimethoxy-4-[(1E)-1-propen-1-yl]benzene, (4E)-4-methyl-5-(4-methylphenyl)-4-pentenal, (2,2-dimethyl-3-[(2Z)-3-methyl-2,4-pentadien-1-yl]oxirane, (2,2-dimethyl-3-[(2E)-3-methyl-2,4-pentadien-1-yl]oxirane, 2-ethyl-1-hexanol, (Z)-3,7-dimethyl-2,6-octadien-1-ol, 1-(3-methyl-1-benzofuran-2-yl)ethenone, (Z)-6-nonen-1-ol, (2E,6Z)-2,6-nonadienal, 1,8-p-menthadien-7-ol, 3-methyl-5-phenyl-1-pentanol, 2,6,6-trimethyl-1,3-cyclohexadiene-1-carbaldehyde, ethyl 2-hydroxybenzoate, 2-(5,5,6-trimethylbicyclo[2.2.1]hept-2-yl)-1-cyclohexanol, 3-(5,5,6-trimethylbicyclo [2.2.1]hept-2-yl)-1-cyclohexanol, 4-(5,5,6-trimethylbicyclo[2.2.1]hept-2-yl)-1-cyclohexanol, 2-(1,7,7-trimethylbicyclo[2.2.1]hept-2-yl)-1-cyclohexanol, 3-methylindole, 8-mercapto-3-p-menthanone, 3,7-dimethyl-3-octanol, 1,3,3-trimethylbicyclo[2.2.1]heptan-2-one, 1-isopropyl-4-methylbicyclo[3.1.0]hexan-3-one, 1-isopropyl-4-methylbicyclo [3.1.0] hexan-3-one, thymol, 6-hexyltetrahydro-2H-pyran-2-one, 3-propylphenol, 2-cyclohexylethyl acetate, octanal, 1,3-nonanediyl diacetate, tetrahydro-3-pentyl-4 (2H)-pyranyl acetate, 5-heptyldihydro-2 (3H)-furanone, 3-butylidene-1-benzo[C] furanone, 3,7-dimethyl-6-octen-1-ol, 3,7-dimethyl-6-octenenitrile, coriander oil, cyclopropylmethyl (3Z)-3-hexenoate, cyclopropylmethyl (3E)-3-hexenoate, 3,7-dimethyl-1-octen-3-ol, cinnamon leaf oil, 4-decanolide, 3,5,6-trimethyl-3-cyclohexene-1-carbaldehyde, 2,4,6-trimethyl-3-cyclohexene-1-carbaldehyde, Lavandin Sumian essential oil, peppermint oil, 3,6,7-trimethyl-2,6-octadienal, petitgrain paraguay essential oil, (2Z)-4,8-dimethyl-2,7-nonadien-4-ol, and a combination thereof

10. The antimicrobial composition of claim 1, wherein the perfume ingredient is selected from the group consisting of: (4E,8E)-4,8-cyclododecadien-1-one, (4E,8Z)-4,8-cyclododecadien-1-one, (4Z,8E)-4,8-cyclododecadien-1-one, (4E)-4-methyl-5-(4-methylphenyl)-4-pentenal, 3,7-dimethyl-6-octenenitrile, 3,7-dimethyl-6-octen-1-ol, (2Z)-4,8-dimethyl-2,7-nonadien-4-ol, 1,3-nonanediyl diacetate, tetrahydro-3-pentyl-4(2H)-pyranyl acetate, (Z)-4-decenal, 3-[4-methyl-3-cyclohexen-1-yl]-1-butanol, undecanal, (2E)-2-ethyl-4-[2,2,3-trimethyl-3-cyclopenten-1-yl]-2-buten-1-ol, 3,7-dimethyl-3-octanol, 2-(5,5,6-trimethylbicyclo[2.2.1]hept-2-yl)-1-cyclohexanol, 3-(5,5,6-trimethylbicyclo[2.2.1]hept-2-yl)-1-cyclohexanol, 4-(5,5,6-trimethylbicyclo[2.2.1]hept-2-yl)-1-cyclohexanol, 2-(1,7,7-trimethylbicyclo[2.2.1]hept-2-yl)-1-cyclohexanol, 2-cyclohexylethyl acetate, 1,3,3-trimethylbicyclo[2.2.1]heptan-2-one, 1-isopropyl-4-methylbicyclo[3.1.0]hexan-3-one, and a combination thereof.

11. The antimicrobial composition of claim 1, wherein the perfume ingredient is selected from the group consisting of: cyclopropylmethyl (3Z)-3-hexenoate or cyclopropylmethyl (3E)-3-hexenoate, 3,6,7-trimethyl-2,6-octadienal, (2Z)-4,8-dimethyl-2,7-nonadien-4-ol, octanal, 5-heptyldihydro-2 (3H)-furanone, 3,7-dimethyl-6-octen-1-ol, 3,7-dimethyl-6-octenenitrile, 3,7-dimethyl-1-octen-3-ol, cinnamon leaf oil, Lavandin Sumian essential oil, 2-cyclohexylethyl acetate, peppermint oil, coriander oil, Petitgrain paraguay essential oil, 3-butylidene-1-benzo[C] furanone, and a combination thereof.

12. The antimicrobial composition according to claim 1, further comprising a solvent and/or a water-miscible co-solvent.

13. The antimicrobial composition according to claim 1, further comprising ingredients selected from the group consisting of colorants, preservatives, viscosifiers, opacifiers, emollients, humectants, antioxidants, gelling agents, gums, chelators, functional polymers, cellulose derivatives, essential oils, electrolytes, and pH adjusters.

14. The antimicrobial composition of claim 1, further comprising a malodor neutralizing system and/or a malodor antagonist system.

15. A consumer product comprising the antimicrobial composition according to claim 1, wherein the consumer product is selected from the group consisting of: a hair care product, a body care product, a skin care product, an oral care product, a female care product, a home care product, a laundry care product, a body cleansing product, shampoo, shower gel, facial cleanser, shaving gel, liquid hand soap, foaming soap, hand sanitizer, soap bar, mouthwash, toothpaste, female hygiene composition, fabric cleanser, carpet cleanser, all-purpose cleanser, dishwashing detergent, fresh produce detergent, deodorant, air fresher, and air disinfectant.

16. A method for eliminating or/and reducing the number of microbes on a surface or body part, the method comprising contacting the surface or body part with the antimicrobial composition of claim 1.
